(19) United States
(12) Patent Application Publication
(10) Pub. No.: US 2019/0249134 A1
(43) Pub. Date: Aug. 15, 2019

(54) CELL CULTURES AND USE THEREOF

(71) Applicant: Boston Biomedical, Inc., Cambridge, MA (US)

(72) Inventors: Chiang Jia Li, Cambridge, MA (US); Harry Rogoff, Wrentham, MA (US); Karen Simon, Haverhill, MA (US)

(21) Appl. No.: 16/130,857

(22) Filed: Sep. 13, 2018

Related U.S. Application Data
(63) Continuation of application No. PCT/US2017/022501, filed on Mar. 15, 2017.
(60) Provisional application No. 62/308,772, filed on Mar. 15, 2016.

(51) Int. Cl. C12N 5/00; G01N 33/50

(52) U.S. Cl. CPC C12N 5/0062; G01N 33/5011; C12N 2537/10; C12N 2513/00; C12N 2533/74; G01N 33/5073

(57) ABSTRACT

The present disclosure provides an alginate-based 3D cell culture as an in vitro system for enriching and maintaining the stemness properties of a cancer cell line and a reliable in vitro system for the development and evaluation of CSC-targeting agents.

The present application claims the benefit of priority under 35 U.S.C. § 119 of U.S. Provisional Patent Application No. 62/308,772 filed Mar. 15, 2016; the content of which is incorporated herein in its entirety by reference.

According to recent statistics, about 14 million people are newly diagnosed as having cancer and about 8 million people die of cancer annually in the world. Anti-tumor agents, surgical operations, radiotherapy, immunotherapy, and the like are widely used to treat cancer. Of these, anti-tumor agents are used most often. Anti-tumor agents usually act on the metabolism of cancer cells. However, such metabolic processes occur in not only cancer cells, but also normal cells. As a result, many anti-tumor agents cause unintended side effects.

Recent studies have discovered the presence of cancer stem cells. Cancer stem cells (CSCs) are a sub-population of cancer cells (found within tumors or hematological cancers) that possess characteristics normally associated with stem cells. These cells are tumorigenic (tumor-forming), in contrast to the bulk of cancer cells, which are non-tumorigenic. In human acute myeloid leukemia the frequency of these cells is less than 1 in 10,000. There is mounting evidence that such cells exist in almost all tumor types. However, as cancer cell lines are selected from a sub-population of cancer cells that are specifically adapted to growth in tissue culture, the biological and functional properties of these cell lines can change dramatically. Therefore, not all cancer cell lines contain cancer stem cells.

CSCs have stem cell properties such as self-renewal and the ability to differentiate into multiple cell types. They persist in tumors as a distinct population and they give rise to the differentiated cells that form the bulk of the tumor mass and phenotypically characterize the disease. CSCs have been demonstrated to be fundamentally responsible for carcinogenesis, cancer metastasis, and cancer reoccurrence. CSCs are also often called tumor initiating cells, cancer stem-like cells, stem-like cancer cells, highly tumorigenic cells, or super malignant cells.

The existence of cancer stem cells has several implications in terms of cancer treatment and therapy. These include disease identification, selective drug targets, prevention of cancer metastasis and recurrence, treatment of cancer refractory to chemotherapy and/or radiotherapy, treatment of cancers inherently resistant to chemotherapy or radiotherapy and development of new strategies in fighting cancer.

The efficacy of cancer treatments are, in the initial stages of testing, often measured by reduction in tumor mass. As CSCs would form a very small proportion of the tumor and have markedly different biologic characteristics than their differentiated progeny, the measurement of tumor mass may not necessarily select for drugs that act specifically on the stem cells. In fact, cancer stem cells are radio-resistant and also refractory to chemotherapeutic and targeted drugs. Normal somatic stem cells are naturally resistant to chemotherapeutic agents—they have various pumps (such as MDR) that efflux drugs, higher DNA repair capability, and have a slow rate of cell turnover (chemotherapeutic agents naturally target rapidly replicating cells). Cancer stem cells, being the mutated counterparts of normal stem cells, may also have similar functions which allow them to survive therapy. In other words, conventional chemotherapies kill differentiated or differentiating cells, which form the bulk of the tumor that are unable to generate new cells. A population of cancer stem cells which gave rise to it could remain untouched and cause a relapse of the disease. Furthermore, treatment with chemotherapeutic agents may only leave chemotherapy-resistant cancer stem cells, so that the ensuing tumor will most likely also be resistant to chemotherapy. Cancer stem cells have also been demonstrated to be resistant to radiotherapy (XRT).

Since surviving cancer stem cells can repopulate the tumor and cause relapse, it would be possible to treat patients with aggressive, non-resectable tumors and refractory or recurrent cancers, as well as prevent the tumor metastasis and recurrence by selectively targeting cancer stem cells. Development of specific therapies targeted at cancer stem cells therefore holds hope for improvement of survival and quality of life of cancer patients, especially for sufferers of metastatic disease. The key to unlocking this untapped potential is the identification and validation of pathways that are selectively important for cancer stem cell self-renewal and survival. Though multiple pathways underlying tumorigenesis in cancer and in embryonic stem cells or adult stem cells have been elucidated in the past, no pathways have been reported for cancer stem cell self-renewal and survival.

Methods of identification and isolation of cancer stem cells have been reported. The methods are used mainly to exploit the ability of CSCs to efflux drugs, or are based on the expression of surface markers associated with cancer stem cells.

CSCs are resistant to many chemotherapeutic agents, therefore it is not surprising that CSCs almost ubiquitously overexpress drug efflux pumps such as ABCG2 (BCRP-1), and other ATP binding cassette (ABC) superfamily members. The side population (SP) technique, originally used to enrich hematopoietic and leukemic stem cells, was first employed to identify CSCs in the C6 glioma cell line. This method, first described by Goodell et al., takes advantage of differential ABC transporter-dependent efflux of the fluorescent dye Hoechst 33342 to define a cell population enriched in CSCs. The SP is revealed by blocking drug efflux with verapamil, so that the SP is lost upon verapamil addition.

More recent studies on target identification suggest a critical role of STAT3 for self-renewal and survival of cancer stem cells. STAT3 is a member of the STAT family which are latent transcription factors activated in response to cytokines/growth factors to promote proliferation, survival, and other biological processes. STAT3 is activated by phosphorylation of a critical tyrosine residue mediated by growth factor receptor tyrosine kinases, Janus kinases, and/or the SRC family kinases, etc. These kinases include but not limited to EGFR, JAKs, ABL, KDR, c-MET, SRC, and HER2. Upon tyrosine phosphorylation, STAT3 forms homo-dimers and translocates to the nucleus, binds to specific DNA-response elements in the promoters of the target genes, and induces gene expression.

In normal cells, STAT3 activation is transient and tightly regulated, lasting from about 30 minutes to several hours. However, STAT3 is found to be aberrantly active in a wide variety of human cancers, including all the major carcinomas as well as some hematologic tumors. STAT3 plays multiple roles in cancer progression. As a potent transcription regulator, it targets genes involved in cell cycle, cell survival, oncogenesis, tumor invasion, and metastasis, such as BCL-XL, c-MYC, CYCLIN D1, VEGF, MMP-2, and SURVIVIN. It is also a key negative regulator of tumor immune surveillance and immune cell recruitment.

Ablating STAT3 signaling by antisense, siRNA, dominant-negative form of STAT3, and/or blockade of tyrosine kinases causes cancer cell-growth arrest, apotosis, and reduction of metastasis frequency in vitro and/or in vivo.

Activation of STAT3 by various cytokines, such as Interleukin 6 (IL-6) has been demonstrated in a number of autoimmune and inflammatory diseases. Recently, it has been revealed that the STAT3 pathway promotes pathologic immune responses through its essential role in generating TH17 T-cell responses. In addition, STAT3 pathway mediated inflammation is the common causative origin for atherosclerosis, peripheral vascular disease, coronary artery disease, hypertension, osteroprorosis, type 2 diabetes, and dementia. Therefore, STAT3 inhibitors may be used to prevent and treat autoimmune and inflammatory diseases as well as the other diseases listed above that are caused by inflammation.

Efforts have also focused on the development of a culture system which can mimic aspects of the tumor microenvironment within the cancer stem cell niche and the use of the culture system to elucidate the mechanisms that underlie the growth and survival of CSCs and to identify compounds that can target and eliminate this highly malignant cell population.

The present disclosure relates to an in vitro system that allows the enrichment and culture of CSCs. In some embodiments, the system allows to assess the efficacy of CSC-targeting drugs reliably.

One aspect of the present disclosure provides a three-dimensional (3D) cell culture. In some embodiments, the 3D cell culture includes at least one culture. In some embodiments, the 3D cell culture includes at least one cross-linker. In some embodiments, the 3D culture includes at least one alginate. In some embodiments, the 3D cell culture is at least one alginate-based cell culture.

In some embodiments, the 3D cell culture includes cells. In some embodiments, the 3D cell culture includes cells with at least one stem-like characteristic. In some embodiments, the cells in the 3D cell culture include stem cells. In some embodiments, the cells include cancer cells. In some embodiments, the cells include another type of cells. In some embodiments, the cells include cancer stem cells (CSC).

In some embodiments, the cells in a cell culture of the present disclosure have increased CSC-related gene expression. In some embodiments, the cells in a cell culture of the present disclosure have at least one alteration in metabolic-related gene expression. In some embodiments, one or both the gene expressions are consistent with changes reported to occur in CSC that help them to maintain their drug resistant and invasive properties.

In some embodiments, the cells in the 3D cell culture are resistant to conventional chemotherapy and/or targeted therapeutics, while remaining sensitive to CSC targeting agents. In some embodiments, the at least one CSC targeting agent is a compound of formula I. In some embodiments, the at least one CSC targeting agent is a compound of formula II.

In some embodiments, the 3D culture enriches and maintains a stemness property of cells. In some embodiments, the cells are cancer cells. In some embodiments, the expression of stemness-associated genes (i) increased and (ii) maintained over multiple passages. In some embodiments, the stemness properties of the cells embedded in the alginate-based 3D cell culture (i) increased and (ii) maintained over multiple passages. In some embodiments, the cells embedded in the alginate-based 3D culture can remain sensitive to CSC-targeting drugs, but become resistant to traditional chemotherapy drugs. Without attempting to limit the scope of the present disclosure and the appended claims, an alginate-based 3D cell culture of the present disclosure can be used to evaluate the efficacy of cancer stem cell-targeting drugs.

Another aspect of the present disclosure provides the 3D cell culture as a culture system in the enrichment and expansion of stem cells, including CSCs. In some embodiments, the alginate-based 3D culture system is used to enrich cells with a stem-like characteristic. In some embodiments, a reliable in vitro system is provided for the development and evaluation of CSC-targeting agents. In some embodiments, the stem-like characteristic of the cells discussed in the present disclosure is maintained over multiple passages of culture. In some embodiments, the stem-like characteristic of the cells discussed in the present disclosure is maintained over many months of continuous culture.

Another aspect of the present disclosure provides methods of preparing a cell culture. In some embodiments, the method includes embedding cells in a 3D cell culture. In some embodiments, the method includes culturing the 3D cell culture for a first period of time. In some embodiments, the first period of time ranges from 12 hours to 30 days. In some embodiments, the first period of time ranges from 1 day to 20 days. In some embodiments, the first period of time ranges from 5 days to 15 days. In some embodiments, the first period of time is about 5 days, about 7 days, about 10 days, about 14 days, or about 15 days.

In some embodiments, the method includes dissolving the cell culture. For example, at least one chelating agent can be added so that at least one crosslinker is extracted by the at least one chelating agent. The at least one chelating agent, in one example, is ethylenediaminetetraacetic acid (EDTA). Similar chelating agents are generally known and can be selected by a person with ordinary skill in the art.

In some embodiments, the method includes separating the cells from the cell culture. For example, the cells can be separated from the dissolved cell culture. Separating cells from a culture is generally known and can be accomplished by a person with ordinary skill in the art.

In some embodiments, the process of embedding and culturing cells in the 3D cell culture is known as a passage. In some embodiments, the process also includes separating cells from the 3D cell culture from the culture for necessary embedding in the subsequent passage. In some embodiments, the method includes repeating the passage. For example, the passage can be repeated once, twice, or more than twice. In some embodiments, the passage is repeated twice, 3 times, 4 times, 5 times, 6 times, 7 times, 8 times, 9 times, 10 times, 11 times, 12 times, 13 times, 14 times, 15 times, or 16 times. Without attempting to limiting the present disclosure or the appended claims by a particular theory or hypothesis, the repetition of passage can enrich cells with a stem-like characteristic, and, in other words, the method can be used to enrich stem cells.

Thus, another aspect of the present disclosure provides methods of using a cell culture of the present disclosure for screening compounds that target the stem-like characteristic (or stemness) of cells. In some embodiments, the method includes screening compounds that target at least one CSC-related gene or its expression pathway. In some embodiments, the method includes screening compounds that target at least one metabolic-related gene expression or its expression pathway. In some embodiments, the method includes screening compounds that target cells that are refractory or become resistant to a conventional drug therapy. In some embodiments, the conventional drug therapy is a conventional oncology drug. In some embodiments, the conventional drug therapy is a conventional chemotherapy. In some embodiments, the conventional drug therapy is a targeted therapy. In some embodiments, the conventional drug therapy is a radiation therapy.

In some embodiments, the method includes culturing a 3D cell culture of the present disclosure in the presence of a compound. In some embodiments, the method includes obtaining an IC₅₀ of a compound from the 3D cell culture. In some embodiments, the method includes obtaining an EC₅₀ of a compound from the 3D cell culture.

The features and advantages of the present disclosure may be more readily understood by those of ordinary skill in the art upon reading the following detailed description and drawings. The detailed description and drawings are merely illustrative of the invention rather than limiting, the scope of the invention being defined by the appended claims and equivalents thereof. It is to be appreciated that certain features of the present disclosure that are, for clarity reasons, described above and below in the context of separate embodiments, may also be combined to form a single embodiment and that various features of the present disclosure that are, for brevity reasons, described in the context of a single embodiment, may also be combined so as to form sub-combinations thereof. Embodiments identified herein as exemplary or preferred are intended to be illustrative and not limiting.

### BRIEF DESCRIPTION OF DRAWINGS

For a more complete understanding of the present disclosure and its features and advantages, reference is now made to the following description, taken in conjunction with the accompanying drawings, in which:

FIG. 1A shows a scheme for embedding cells in an alginate-based 3D cell culture;

FIG. 1B shows an exemplary enrichment of sphere-forming cells in the alginate-based 3d cell culture within passages according to some embodiments of the present disclosure;

FIG. 1C shows a graphic summary of the increase in the population of sphere forming cells within passages according to some embodiments of the present disclosure;

FIG. 2A shows exemplary micrographs of spheres increase in size from 7 days (Left Image) to 14 days (Right Image) of culture in the alginate-based 3D cell culture according to some embodiments of the present disclosure;

FIG. 2B shows exemplary expressions of CSC-related and glucose metabolism-related genes of A549 cells cultured in an alginate-based 3D cell culture (Passage 5) and a conventional 2D culture using PCR-based gene arrays according to some embodiments of the present disclosure (for illustrative purpose, each graph includes two vertical lines, representing that the area to the left of the first vertical line is where the expression is downregulated, the area between the first and the second vertical lines is where no change is observed in the expression, and the area to the right of the second vertical line is where the expression is upregulated);

FIG. 2C shows exemplary expressions of CSC-related and glucose metabolism-related genes in long-term cultures of A549 in an alginate-based 3D cell culture (Passage 12) and a conventional 2D culture (for illustrative purpose, each graph includes two vertical lines, representing that the area to the left of the first vertical line is where the expression is downregulated, the area between the first and the second vertical lines is where no change is observed in the expression, and the area to the right of the second vertical line is where the expression is upregulated);

FIG. 3A shows selected stemness-related and glycolysis-related genes with increased expression in an alginate-based 3D cell culture according to some embodiments of the present disclosure;

FIG. 3B shows that exemplary expression of CSC-related, glucose metabolism-related and hypoxia-related genes in an alginate-based cell culture and a conventional 2D culture were validated using qPCR;

FIG. 4A shows a summary of IC₅₀ of A549 Cells in an alginate-based 3D cell culture and a conventional 2D cell culture according to some embodiments of the present disclosure;

FIGS. 4B and 4C show the exemplary fold change resistance of selected chemotherapy, targeted therapy and stemness-targeting drugs in an alginate-based 3D cell culture according to some embodiments of the present disclosure (The fold change resistance is defined by the IC₅₀ value of an agent in the alginate-based 3D cell culture divided by the IC₅₀ value of the agent in the conventional 2D culture); and

FIG. 5 shows exemplary micrographs (7 days after the drug treatment) that represent the drug response of A549 cellular spheroids to BBI-608, BBI-503, sunitinib, doxorubicin, and gemcitabine in an alginate-based 3D cell culture according to some embodiments of the present disclosure.

Unless specifically stated otherwise, references made in the singular may also include the plural. For example, “a” and “an” may refer to either one or one or more.

When a range of values is listed herein, it is intended to encompass each value and sub-range within that range. For example, “1-5 mg” is intended to encompass 1 mg, 2 mg, 3 mg, 4 mg, 5 mg, 1-2 mg, 1-3 mg, 1-4 mg, 1-5 mg, 2-3 mg, 2-4 mg, 2-5 mg, 3-4 mg, 3-5 mg, and 4-5 mg.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

As used herein, the term “alginate” refers to a salt, solution, or hydrogel of a polysaccharide biocopolymer comprised of guluronic acid and mannuronic acid. In some embodiments, the at least one alginate used in this disclosure can be any water-soluble salt of alginic acid. In some embodiments, the at least one alginate is sodium alginate. In some embodiments, the at least one alginate concentration in aqueous solution can be up to about 10 percent by weight. In some embodiments, the at least one alginate concentration is between about 0.5 percent and about 2.0 percent by weight. The average molecular weight of the at least one alginate can be between about 80,000 and about 200,000 Daltons. In some embodiments, the aqueous alginate solution is prepared with deionized water, diluted brine, or basal medium that is generally used in culturing cells. In some embodiments, an alginate hydrogel is formed by adding at least one crosslinker into an aqueous alginate solution. Alginate hydrogels are included in the term “alginate.” In some embodiments, the at least one alginate is an alginate-based three dimensional system. In some embodiments, the at least one alginate is an alginate-based 3D cell culture.

As used herein, the term “crosslinker” refers to at least one gelling agent that can convert at least one alginate in a low viscosity state to a high viscosity gel. In some embodiments, the at least one crosslinker is an ion. In some embodiments, the at least one crosslinker is a metallic ion. In some embodiments, the at least one crosslinker is a divalent or multivalent metallic ion. In some embodiments, the at least one crosslinker is Pb²⁺, Ba²⁺, Fe³⁺, Al³⁺, Cu²⁺, Cd²⁺, Ho³⁺, Ca²⁺, Zn²⁺, Co²⁺, Ni²⁺, Mn²⁺, and Mg²⁺. In some embodiments, the at least one crosslinker is Ca²⁺. In some embodiments, the at least one crosslinker is a nonmetallic ion. In some embodiments, the at least one crosslinker is a divalent or multivalent nonmetallic ion.

As used herein, the term “cell” refers to any prokaryotic or eukaryotic cell. Such cells include, for example, bacterial cells (such as E. coli), insect cells, yeast cells, or mammalian cells (such as Chinese hamster ovary cells (CHO) cells, COS cells, VERO cells, BHK cells, HeLa cells, Cv1 cells, MDCK cells, 293 cells, 3T3 cells, or PC12 cells). Other exemplary cells include cells from the members of the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Schizosaccharomyces, Yarrowia, or Streptomyces. In some embodiments, the cell is a mammalian cell. In some embodiments, the cell is a human cell. In some embodiments, the cell is a cancer cell or a tumor cell. In certain instances, the cells can be transformed or transfected with one or more expression vectors or viral vectors.

As used herein, the term “cancer” in a subject refers to the presence of cells possessing characteristics typical of cancer-causing cells, such as uncontrolled proliferation, immortality, metastatic potential, rapid growth and proliferation rate, or/and certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, colon cancer, rectal cancer, cancer of the anal region, stomach cancer, gastric cancer, gastrointestinal cancer, uterine cancer, carcinoma of the fallopian tubes, carcinoma of the endometrium, carcinoma of the vagina, carcinoma of the vulva, Hodgkin's Disease, esophageal cancer, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and both the exemplified cancers and the general terms are included in the term “cancer.” For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

Also included within the term “cancer” is “solid tumor.” As used herein, the term “solid tumor” refers to those conditions, such as cancer, that form an abnormal tumor mass, such as sarcomas, carcinomas, and lymphomas. Examples of solid tumors include, but are not limited to, non-small cell lung cancer (NSCLC), neuroendocrine tumors, thyomas, fibrous tumors, metastatic colorectal cancer (mCRC), and the like. In some embodiments, the solid tumor disease is an adenocarcinoma, squamous cell carcinoma, large cell carcinoma, and the like.

Thus, as used herein, the term “cancer cell” refers to a cell originated or obtained from any one of the cancers discussed herein.

As used herein, “cancer stem cell” (“CSC”) or “cancer stem cells” (“CSCs”) refer to a population of cancer cells that have self-renewal capability and are tumorigenic. They are also called “cancer initiating cells,” “tumor initiating cells,” “cancer stem-like cells,” “stem-like cancer cells,” “aggressive cancer cells,” and “super malignant cancer cells,” etc. The methods of isolating these cells include but are not limited to enrichment by their ability of efflux Hoechst 33342, enrichment of surface markers such as CD133, CD44, and others, and enrichment by their tumorigenic property.

As used herein, the term “cancer stemness inhibitor” refers to a compound that is capable of suppressing CSCs. Without being limited to any particular theory, a cancer stemness inhibitor can target or/and inhibit multiple pathways involved in cancer stem cell's stem-like characteristics. For example, the multiple pathways can involve STAT3, β-CATENIN, NANOG, TCF4, STK33, and the like. Cancer stemness inhibitors can be a small molecule or a biologic (including a sugar, a peptide, a protein, a nucleic acid, or a combination thereof). In some embodiments, a cancer stemness inhibitor of the present disclosure is a compound of formula I or formula II.

As used herein, the term “subject” refers to human and non-human animals, including veterinary subjects. The term “non-human animal” includes all vertebrates, e.g., mammals and non-mammals, such as non-human primates, mice, rabbits, sheep, dogs, cats, horses, cows, chickens, amphibians, and reptiles. In some embodiments, the subject is a human and may be referred to as a patient.

As used herein, the terms “treat,” “treating,” or “treatment” refer, in some embodiments, to an action to obtain a beneficial or desired clinical result including, but not limited to, alleviation or amelioration of one or more signs or symptoms of a disease or condition, diminishing the extent of disease, stabilization (i.e., not worsening) of the state of disease, amelioration or palliation of the disease state, diminishing rate of or time to progression, remission (whether partial or total), whether detectable or undetectable, or/and prevention of a disease or condition. “Treatment” can also mean prolonging survival as compared to expected survival in the absence of treatment. Treatment does not need to be curative and can be an action to administer a CSC targeting agent to a healthy human who has not developed a disease, for example, to delay or avoid the onset of a disease. Sometimes, this is also referred to as “prevent,” “preventing,” or “prevention.”

As used herein, the term “effective amount” of an active agent refers to an amount sufficient to elicit the desired biological response. As will be appreciated by those of ordinary skill in this art, the effective amount of a CSC targeting agent may vary depending on such factors as the desired biological endpoint, the pharmacokinetics of the compound, the disease being treated, the mode of administration, or/and the patient.

An “effective amount” of an anti-cancer agent in reference to decreasing cancer cell growth means an amount capable of decreasing, to some extent, the growth of some cancer or tumor cells. The term includes an amount capable of invoking a growth inhibitory, cytostatic and/or cytotoxic effect, and/or apoptosis of the cancer or tumor cells.

A “therapeutically effective amount” in reference to the treatment of cancer, means an amount capable of invoking one or more of the following effects: (1) inhibition, to some extent, of cancer or tumor growth, including slowing down growth or complete growth arrest; (2) reduction in the number of cancer or tumor cells; (3) reduction in tumor size; (4) inhibition (i.e., reduction, slowing down, or complete stopping) of cancer or tumor cell infiltration into peripheral organs; (5) inhibition (i.e., reduction, slowing down, or complete stopping) of metastasis; (6) enhancement of anti-tumor immune response, which may, but is not required to, result in the regression or rejection of the tumor, or/and (7) relief, to some extent, of one or more symptoms associated with the cancer or tumor. The therapeutically effective amount may vary according to factors such as the disease state, age, sex, and weight of the individual and the ability of one or more anti-cancer agents to elicit a desired response in the individual. A “therapeutically effective amount” is also one in which any toxic or detrimental effects are outweighed by the therapeutically beneficial effects.

The terms “treating cancer,” “treatment of cancer,” or an equivalent thereof mean to decrease, reduce, or inhibit the replication of cancer cells; decrease, reduce or inhibit the spread (formation of metastases) of cancer; decrease tumor size; decrease the number of tumors (i.e., reduce tumor burden); lessen or reduce the number of cancerous cells in the body; prevent recurrence of cancer after surgical removal or other anti-cancer therapies; or/and ameliorate or alleviate the symptoms of the disease caused by the cancer.

The terms “combination” or “combinatorial,” as used herein, mean the administration of at least two different agents to treat a disorder, condition, or symptom, e.g., a cancer condition. Such combination therapy may involve the administration of one agent before, during, and/or after the administration of a second agent. The compounds, products, and/or pharmaceutical compositions described herein and the second agent can be administered to a subject, for example, a human subject, in the same pharmaceutical composition. Alternatively, the compounds, products, and/or pharmaceutical compositions described herein and the second agent can be administered concurrently, separately, or sequentially to a subject in separate pharmaceutical compositions. The compounds, products, and/or pharmaceutical compositions described herein and the second agent may be administered to a subject by the same or different routes of administration. In some embodiments, a combination of the present disclosure comprises an effective amount of the compounds, products, and/or pharmaceutical compositions described herein and an effective amount of at least one second agent (e.g., prophylactic or therapeutic agent). For example, the at least one second agent can have a different mechanism of action than the compounds, products, and/or pharmaceutical compositions described herein. In some embodiments, a combination of the present disclosure improves the prophylactic or therapeutic effect of the compounds, products, and/or pharmaceutical compositions described herein and of the second agent by functioning together to have an additive or synergistic effect. In some embodiments, a combination of the present disclosure reduces the side effects associated with the second therapy. The administrations of the agents (including a compound or composition of the present disclosure or a second agent) may be separated in time by up to several weeks, in some embodiments, within 48 hours, and, in other embodiments, within 24 hours.

The terms “synergy” and “synergistic” mean that the effect achieved with the compounds used together is greater than the sum of the effects that results from using the compounds separately, i.e., greater than what would be predicted based on the two active ingredients administered separately. A synergistic effect may be attained when the compounds are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anticancer effect denotes an anticancer effect which is greater than the predicted purely additive effects of the individual compounds of the combination.

As used in the present disclosure, the term “stem-like characteristic” or “stemness” refers a characteristic that can be observed in or associated with a stem cell. For example, one of the stem-like characteristics can be chosen from an ability of differentiating into different cell types, an ability of self-renewal, an ability of survival under certain conditions (including hypoxia conditions), an ability to resist chemotherapeutic agents, and an expression of a wide variety of markers. In some embodiments, the “stem-like characteristic” or “stemness” refers to cells that have various pumps (such as MDR) that efflux drugs, a higher DNA repair capability, and/or a relatively slower rate of cell turn over. When used in connection with cancer cells, the term “stem-like characteristic” or “stemness” can be used also to refer to a cancer cell that is carcinogenetic, metastatic, or/and refractory or resistant to a conventional cancer treatment (including chemotherapy, target therapy, radiation therapy). In some embodiments, the term can also indicate the expression of certain CSC-related gene, including one or more of ABGC2, β-CATENIN, CD34, CD38, DDR1, KLF4, MUC1, NANOG, POU5F1, STAT3, STK33, SOX2, or TCF4. In some embodiments, the term can also indicate the expression of one or more of certain cancer stem cell pathway kinases (CSCPK).

One aspect of the present disclosure provides a 3D cell culture. In some embodiments, the 3D cell culture includes at least one alginate. In some embodiments, the 3D cell culture includes at least one crosslinker. Thus, the term “3D cell culture” used in the present disclosure can be substituted with the term “alginate-based 3D cell culture,” which is an embodiment of such term; and in some embodiments, the term “alginate-based 3D cell culture” can be substituted with the term “3D cell culture,” a broader term, without going beyond the scope of this disclosure and/or the appended claims.

In some embodiments, the 3D cell culture is used to culture cells. In some embodiments, the 3D cell culture is used to culture human cells. In some embodiments, the 3D cell culture is used to culture cancer cells. In some embodiments, the 3D cell culture is used to culture cancer stem cells.

Without being limited to any particular theory or hypothesis, alginate is found to be an ideal system for CSC because of its inertness and ability to provide a hypoxic environment essential for maintaining the stem-like properties of these cells. In some embodiments, cells cultured in the alginate-based 3D cell culture have at least one increased CSC-related gene expression. In some embodiments, cells cultured in the alginate-based 3D cell culture have at least one alteration in metabolic-related gene expression. In some embodiments, one or both the gene expressions are consistent with changes reported to occur in CSC that help them to maintain their drug resistant and invasive properties.

Another aspect of the present disclosure provides a 3D cell culture as a culture system in the enrichment and expansion of CSCs. In some embodiments, the 3D cell culture is used as an in vitro system for enriching and maintaining a stemness characteristic of a cancer cell line. In some embodiments, one or more of the stem-like characteristics of the cells cultured in the 3D cell culture are maintained over multiple passages of continuous culture. In some embodiments, one or more of the stem-like characteristics of the cells cultured in the 3D cell culture are maintained over many months of continuous culture. In some embodiments, the CSC cultures in the 3D cell culture are resistant to at least one conventional chemotherapy. In some embodiments, the CSC cultures in the 3D cell culture are resistant to at least one targeted therapeutic. In some embodiments, the CSC cultures in the 3D cell culture are sensitive to at least one CSC targeting agent. In some embodiments, the at least one CSC targeting agent is a compound of formula I. In some embodiments, the at least one CSC targeting agent is a compound of formula II.

Another aspect provides a method of preparing a cell culture of the present disclosure. In some embodiments, the cell culture is one with the stem-like characteristics. In some embodiments, the cell culture is a stem cell culture. In some embodiments, the cell culture is a cancer stem cell culture. In some embodiments, the cell culture is a cancer cell culture with one or more stem-like characteristics.

In some embodiments, the method includes embedding cells in a 3D cell culture of the present disclosure. In some embodiments, the method includes culturing cells in a 3D cell culture of the present disclosure. In some embodiments, the method includes culturing cells in the 3D cell culture for a period of time ranging from 1 day to 30 days. In some embodiments, the method includes culturing cells in the 3D cell culture for a period of time ranging from 1 day to 20 days. In some embodiments, the method includes culturing cells in the 3D cell culture for a period of time ranging from 1 day to 15 days. In some embodiments, the method includes culturing cells in the 3D cell culture for a period of time ranging from 5 days to 15 days. In some embodiments, the method includes culturing cells in the 3D cell culture for 5 days, 6 days, 7 days, 8 days, 9 days, 10 days, 11 days, 12 days, 13 days, 14 days, or 15 days.

In some embodiments, the method includes embedding cells in an alginate-based 3D cell culture of the present disclosure. In some embodiments, the method includes culturing cells in an alginate-based 3D cell culture of the present disclosure. In some embodiments, the method includes culturing cells in the alginate-based 3D cell culture for a period of time ranging from 1 day to 30 days. In some embodiments, the method includes culturing cells in the alginate-based 3D cell culture for a period of time ranging from 1 day to 20 days. In some embodiments, the method includes culturing cells in the alginate-based 3D cell culture for a period of time ranging from 1 day to 15 days. In some embodiments, the method includes culturing cells in the alginate-based 3D cell culture for a period of time ranging from 5 days to 15 days. In some embodiments, the method includes culturing cells in the alginate-based 3D cell culture for 5 days, 6 days, 7 days, 8 days, 9 days, 10 days, 11 days, 12 days, 13 days, 14 days, or 15 days.

In some embodiments, the method includes dissolving a 3D cell culture embedded with cells. In some embodiments, the method includes adding a dissolution medium having at least one chelating agent. Without limiting the scope of the present disclosure and the appended claims, the at least one chelating agent in the dissolution medium can chelate the at least one crosslinker in the alginate-based 3D cell culture and, as a result, the 3D cell culture can be dissociated or dissolved. For example, the at least one chelating agent can be ethylenediaminetetraacetic acid (EDTA).

In some embodiments, the method includes separating cells from an alginate-based medium. A person with ordinary skill in the art can select a method for separating cells from medium. For example, the cells can be separated by the centrifuge.

In some embodiments, the method includes embedding the resulting cells in an alginate-based 3D cell culture as discussed in the present disclosure. In some embodiments, the method includes culturing the embedded cells in the alginate-based 3D culture. In some embodiments, the embedding and culturing cells in an alginate-based 3D cell culture, optionally with dissolving the alginate-based 3D cell culture and separating the resulting cells from the culture, is sometimes referred to as a “passage.” In some embodiments, the passage is repeated more than once. In some embodiments, the passage is repeated twice. In some embodiments, the passage is repeated three times. In some embodiments, the passage is repeated four times. In some embodiments, the passage is repeated five times. In some embodiments, the passage is repeated six times. In some embodiments, the passage is repeated seven times. In some embodiments, the passage is repeated eight times. In some embodiments, the passage is repeated nine times. In some embodiments, the passage is repeated ten times. In some embodiments, the passage is repeated more than ten times. By doing so, in some embodiments, cells with high stem-like characteristics can be enriched. In some embodiments, stem cells can be enriched. In some embodiments, the stem cells are cancer stem cells. In some embodiments, cell cultures with cells having high stem-like characteristics are prepared. In some embodiments, cell cultures with cancer stem cells are prepared.

Another aspect of the present disclosure provides a 3D cell culture as a reliable in vitro system for the development and evaluation of CSC targeting agents. In some embodiments, the 3D cell culture is an alginate-based 3D cell culture. In some embodiments, the 3D cell culture is used to evaluate and identify CSC targeting agents.

In some embodiments, the 3D cell culture is used to investigate changes in at least one of the stem-like characteristics of a cancer cell line. In some embodiments, the 3D cell culture is used to investigate changes in at least one of the stem-like characteristics of the cancer cell line when they are cultured in the 3D culture system in the presence of a compound. In some embodiments, a cancer cell line is cultured in the 3D cell culture in the presence of a compound. In some embodiments, a cancer cell line is cultured in the 3D cell culture in the absence of any compound. In some embodiments, a cancer cell line is cultured in the 3D cell culture in the presence of at least one known CSC targeting agent.

In some embodiments, at least one of the stem-like characteristics of the cancer cell line is measured. In some embodiments, the at least one of the stem-like characteristics is STAT3, β-CATENIN, NANOG, TCF4, STK33, or the like. In some embodiments, the at least one of the stem-like characteristics in the cell line cultured in the 3D cell culture in the presence of a compound is compared against that in the cell line cultured in the 3D cell culture in the absence of any compound or in the presence of at least one known CSC-targeting agent.

Another aspect provides at least one CSC-targeting agent that is identified by using the 3D cell culture of the present disclosure. In some embodiments, at least one CSC targeting agent is a cancer stemness inhibitor. In some embodiments, the at least one CSC targeting agent is 2-acetylnaphtho[2,3-b]furan-4,9-dione, a prodrug thereof, a pharmaceutically acceptable salt thereof, or a pharmaceutically acceptable solvate thereof. In some embodiments, the at least one CSC targeting agent is chosen from compounds having formula I:

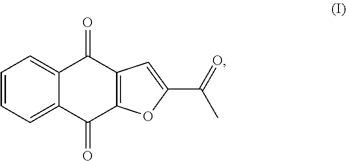 [SMILES: CC(=O)c1cc2c(o1)C(=O)c1ccccc1C2=O]

prodrugs thereof, pharmaceutically acceptable salts thereof, and pharmaceutically acceptable solvates thereof. In some embodiments, a compound is prepared, for example, by using Examples 8-11 in U.S. Pat. No. 9,084,766, the contents of which are incorporated by reference herein in its entirety. In some embodiments, 2-acetylnaphtho[2,3-b]furan-4,9-dione, a compound having formula I, and the compound prepared by using Examples 8-11 in U.S. Pat. No. 9,084,766 can be used interchangeably.

In some embodiments, the at least one CSC-targeting agent is chosen from compounds of formula II:

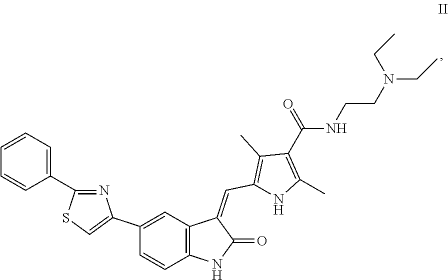 [SMILES: CCN(CC)CCNC(=O)c1c(C)[nH]c(/C=C2\C(=O)Nc3ccc(-c4csc(-c5ccccc5)n4)cc32)c1C]

prodrug thereof, pharmaceutically acceptable salts thereof, and pharmaceutically acceptable solvates thereof.

In some embodiments, the compound of formula II is prepared, for example, according to U.S. Pat. No. 8,299,106, the contents of which are incorporated by reference herein in its entirety. In some embodiments, the compound having formula II and the compound prepared according to U.S. Pat. No. 8,299,106 can be used interchangeably.

Another aspect of the present disclosure provides a combination identified by using a 3D cell of the present disclosure.

In some embodiments, the at least one CSC targeting agent or the combination is for treating cancer. In some embodiments, the at least one CSC targeting agent or the combination is for treating a disease or condition that is associated with STAT3. In some embodiments, the disease or condition is associated with aberrant STAT3 pathway activity. In some embodiments, the disease or condition is associated with expression of activated STAT3. In some embodiments, the disease or condition is an autoimmune disease, an inflammatory disease, inflammatory bowel diseases, arthritis, autoimmune demyelination disorder, Alzheimer's disease, stroke, ischemia reperfusion injury, or multiple sclerosis.

Without being limited to any particular theory, a combination of the present disclosure can enhance the therapeutic activity (for example, the anticancer activity) of at least one CSC targeting agent or/and at least one second agent or/and reduce side effects of the Compound or the at least one second agent. Further, synergistic effects can be observed in a combination of the present disclosure. In some embodiments, the combination includes at least one CSC targeting agent and at least one second agent. In some embodiments, the combination includes a composition disclosed herein and at least one second agent.

In some embodiments, the cancer may be refractory. In some embodiments, the cancer may be recurrent. In some embodiments, the cancer may be metastatic. In some embodiments, the cancer may be associated with overexpression of STAT3. In some embodiments, the cancer may be associated with expression of activated STAT3. In some embodiments, the cancer may be associated with nuclear β-CATENIN overexpression.

Examples, tables, and figures are provided to facilitate a person with ordinary skill in the art to understand this disclosure and appreciate the appended claims. As such, they are not used to limit the scope of the present disclosure and the appended claims. It should be noted that compound names shown in the following reference examples and Examples do not always follow the IUPAC nomenclature. It should be noted that although abbreviations are sometimes used to simplify a description, these abbreviations are defined in the same manner as the above descriptions.

The present disclosure is further illustrated by the following examples and drawings. The examples and drawings are merely illustrative of the invention, rather than limiting, and the scope of the invention is defined by the appended claims and equivalents thereof.

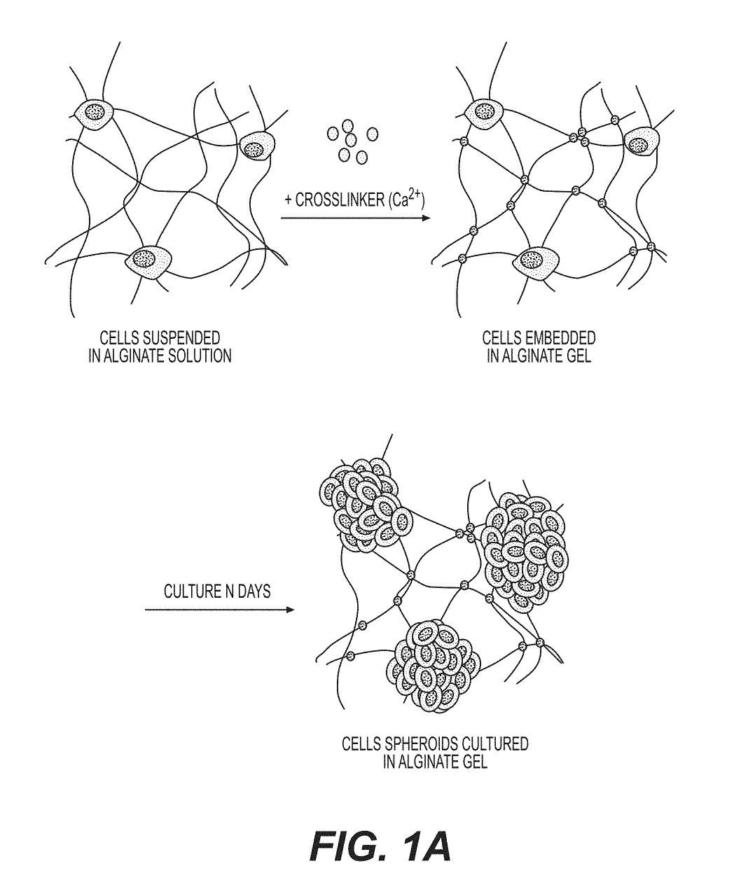

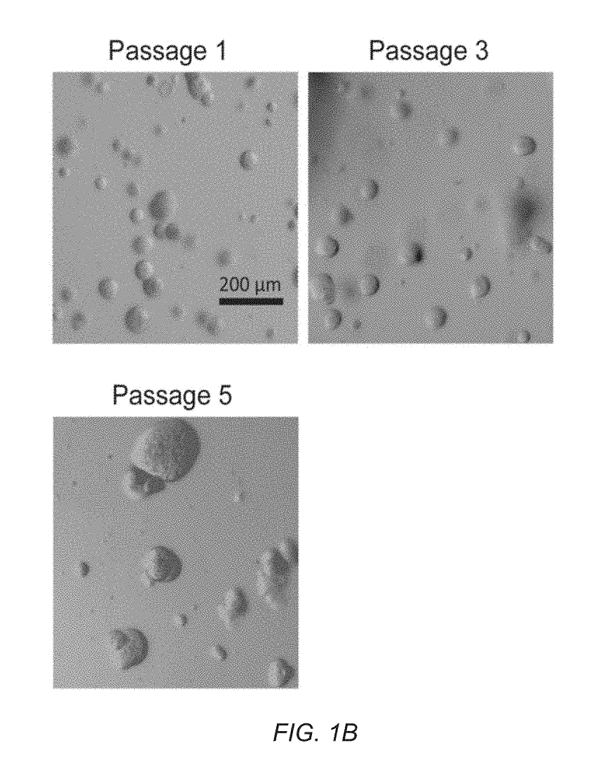

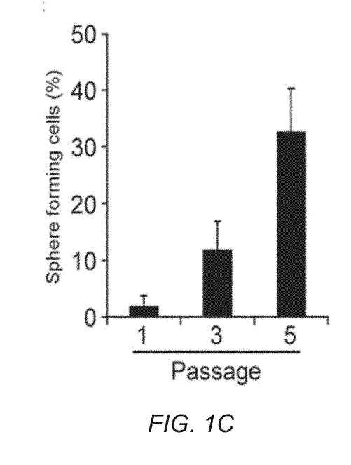

Alginate-based hydrogels are known to be inert, and with the appropriate thickness can provide a hypoxic microenvironment to cells; these two features make alginate gels a suitable matrix for culturing cells with stem-like properties. FIG. 1A describes a scheme for embedding cells in an alginate-based 3D cell culture as single cells. In some embodiments, cells are embedded in the alginate-based 3D cell culture by mixing a suspension of cells in an aqueous solution of sodium alginate with an aqueous solution of calcium chloride. Upon mixing, the glucoronate groups of the alginate can chelate the calcium ions to form a cross-linked gel matrix. The suspended cells can therefore be embedded within the matrix upon the formation of the alginate gel. The embedded cells can be recovered for analyses and subsequent passage by adding a solution of EDTA which chelates calcium. This process can disrupt the calcium-glucoranate crosslinks and thus dissolve the gel and releases the cells from the matrix.

In some embodiments, cells were suspended in an aqueous solution of alginate and gelled by adding aqueous CaCl₂. In some embodiments, the cells are seeded at a density of 5×10⁴ cells/mL. In some embodiments, the embedded cells form multicellular spheroids after 7-14 days of culture. In some embodiments, cells are passaged by de-gelling (dissociating or dissolving) the alginate-based 3D cell culture with a solution of EDTA, and treating the spheroids with Accutase to redisperse into single cells.

FIG. 1B shows the exemplary enrichment of sphere-forming cells in an alginate-based 3D cell culture within passages. As shown in FIG. 1B, in some embodiments, the spheroids increased in size, and decreased in number from Passage 1 to Passage 5. Without limiting the scope of the present disclosure or the appended claims with any particular theory or hypothesis, the change in sphere size and numbers can indicate that the number of sphere-forming cells increases with the passage number.

FIG. 1C includes several graphs summarizing the increase in the population of sphere forming cells within passages. As shown in the graph in FIG. 1C, the percentage of sphere-forming cells in Passages 1, 3, and 5 increased continuously from 2±2% to 12±5% to 33±8%, respectively. Without limiting the scope of the present disclosure or the appended claims with any particular theory or hypothesis, these results demonstrated that culturing cells in an alginate-based 3D cell culture can enrich for sphere-forming cells, and further suggest that an alginate-based 3D cell culture of the present disclosure can enrich for cells with stem-like properties.

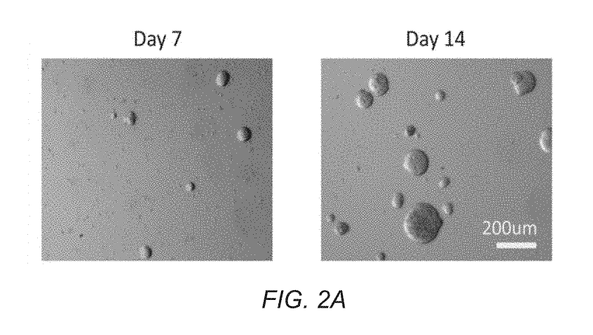

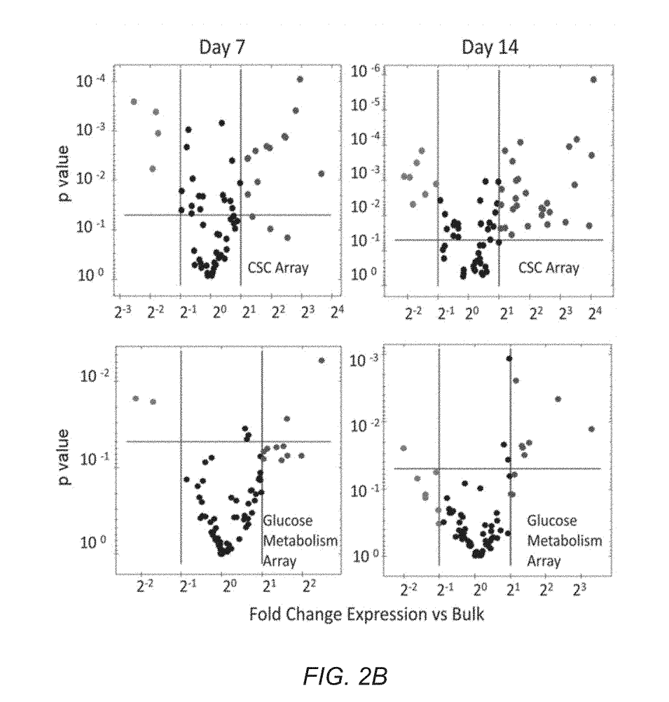

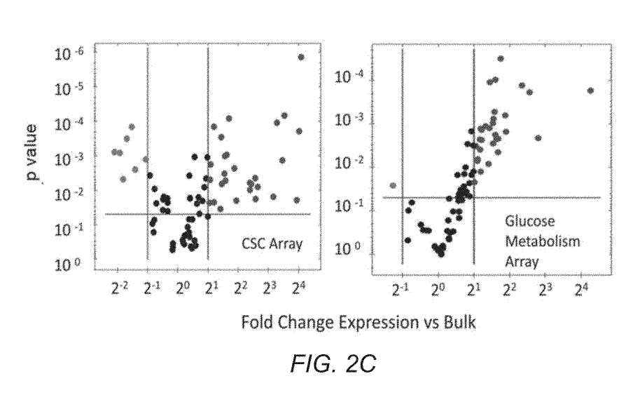

FIG. 2A shows several micrographs of sphere increase in size from 7 days (Left Image) to 14 days (Right Image) of in an alginate-based 3D cell culture of the present disclosure. As shown in FIG. 2A, the alginate embedded cells form spheroids that can increase by at least 50% in diameter between 7 and 14 days.

Without limiting the scope of the present disclosure or the appended claims with any particular theory or hypothesis, since an increase in the size of the spheroids could further limit the mass transport of oxygen and other nutrients and thus may create a more hypoxic microenvironment within the inner core of the spheroids, the stemness properties of cells can be further enriched with time. To verify whether the expression of stemness and metabolic genes can be altered when cultured in an alginate-based 3D cell culture, A549 cells were harvested from a cell culture after 7 and 14 days of culture and analyzed by using qPCR gene arrays. FIG. 2B shows the expression of CSC-related genes of A549 cells cultured in the alginate-based 3D cell culture (Passage 5) and 2D culture using PCR-based gene arrays. As shown in FIG. 2B, the CSC gene arrays verified that the stemness of cells in the alginate-based 3D cell culture increased between 7 and 14 days of culture compared to cells cultured in a 2D conventional culture. Continuing referring to FIG. 2B, while the number of upregulated metabolism-related genes did not increase with time (at Passage 9), the number of upregulated genes that are statistically-significant (i.e., p-value <0.05) increased with time. Without limiting the scope of the present disclosure or the appended claims by any particular theory or hypothesis, these data demonstrated that cells cultured in an alginate-based 3D cell culture of the present disclosure can be enriched with stemness-high cells compared to cells cultured under a traditional 2D culture.

In some embodiments, 3D cell cultures of the present disclosure promote and enrich stem-like properties of cancer cells and pluripotency of stem cells. To evaluate the ability of the alginate-based 3D cell culture to maintain long-term stemness of A549 cells, the stemness and metabolic gene in cells at passage 12, or approximately 6 months of continuous culture, were analyzed using qPCR arrays.

FIG. 2C shows the expression of CSC-related and glucose metabolism-related genes in long-term cultures of A549 in an alginate-based 3D cell culture of the present disclosure (Passage 12) and a conventional 2D culture. As shown in FIG. 2C, the CSC gene profile of A549 cells after 14 days of culture remained similar between Passages 5 and 12, while the upregulation of metabolism-related genes dramatically increased from Passages 5 to 12. Without limiting the scope of the present disclosure or the appended claims with any particular theory or hypothesis, these data demonstrated that an alginate-based 3D cell culture of the present disclosure can maintain the stemness of cells in long-term culture.

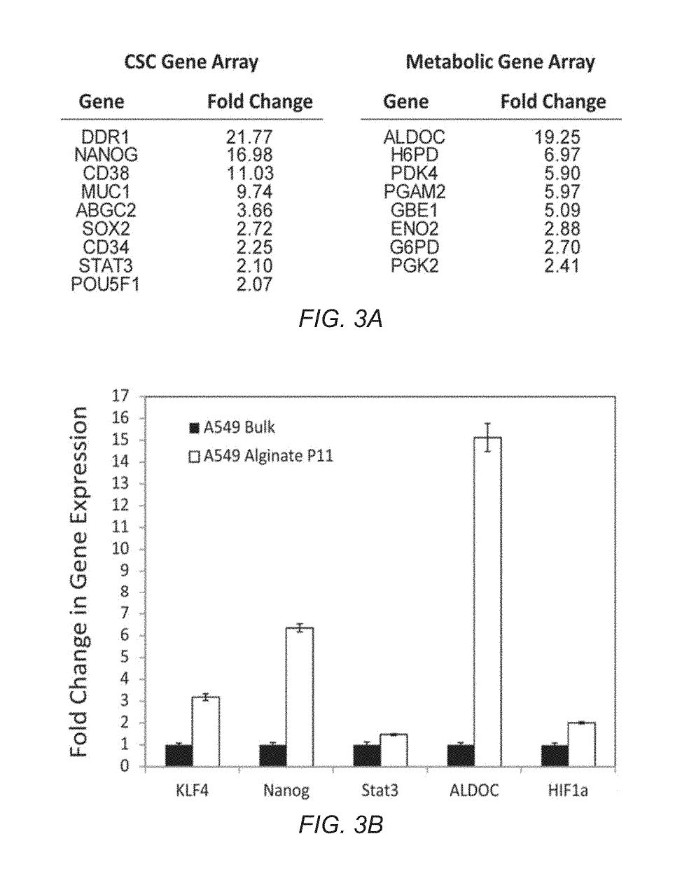

To validate the results from the gene array experiments, the expression of a number of stemness-related (STAT3, KLF4, NANOG, glycolysis-related (ALDOC), and hypoxia-related genes (HIF1α) from cDNA of cells cultured in alginate-based 3D cell cultures were compared to those from cells in conventional 2D cultures. FIG. 3A shows the increased expression of several selected stemness-related and glycolysis-related genes of cells in the alginate-based 3D cell cultures. As shown in FIG. 3A, the fold changes of these genes range from about 2 to about 20.

FIG. 3B shows that the expression of CSC-related, glucose metabolism-related and hypoxia-related genes in the alginate-based 3D and the conventional 2D cell cultures were validated using qPCR. As shown in FIG. 3B, the stemness-related genes STAT3, KLF4, and NANOG were upregulated between about 2- and about 7-fold. Continuing to refer to FIG. 3B, the glycolysis-related gene aldolase C (ALDOC), a gene encoding for the glycolytic enzyme for the reversible aldol cleavage of fructose 1,6-bisphosphate and fructose-1-phosphate to dihydroxyacetone phosphate and either glyceraldehyde 3-phosphate or glyceraldehyde, respectively, was upregulated by about 16-fold. Continuing referring to FIG. 3B, the hypoxia-related gene HIF1α was upregulated by about 2-fold. Without limiting the scope of the present disclosure or the appended claims with any particular theory or hypothesis, these results indicated that, in addition to increased expression of stemness-related genes, genes associated with increased stemness properties such as glycolysis-related genes and hypoxia-related genes also increased in cells cultured in alginate-based 3D cell cultures of the present disclosure.

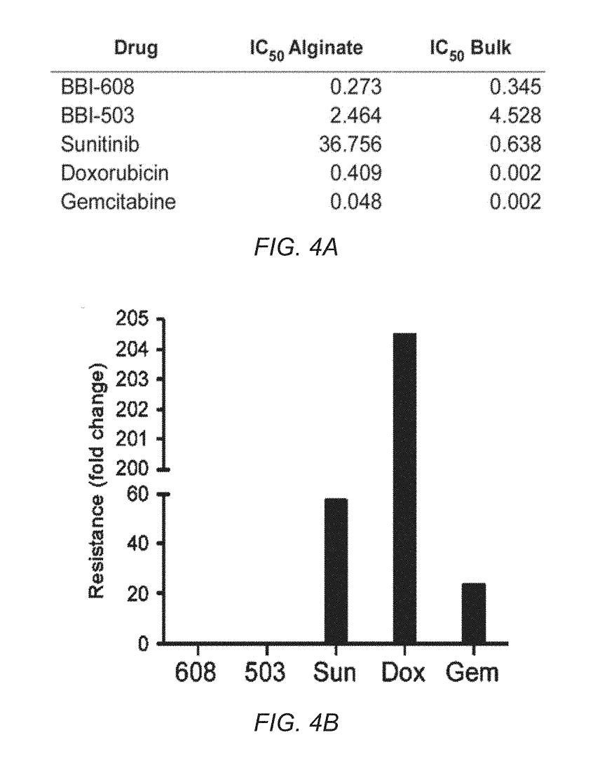

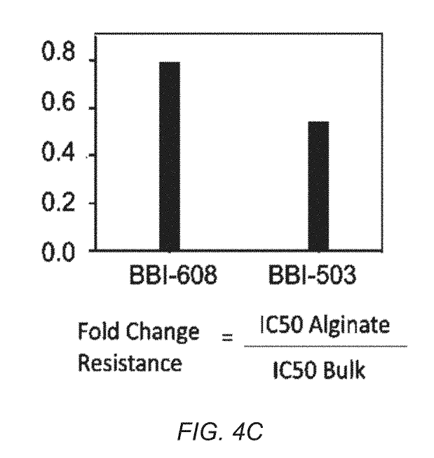

Without limiting the scope of the present disclosure or appended claims with any particular theory or hypothesis, since the stemness is associated with a resistance to chemotherapy, cells cultured in an alginate-based 3D cell culture may be more resistant to chemotherapy drugs and more sensitive to stemness-targeting drugs than cells cultured in traditional 2D cultures. To evaluate the response of cells to drugs, the IC₅₀ values of cells in both the alginate-based 3D and the conventional 2D cell cultures were determined. FIG. 4A summarizes the IC₅₀ of the CSC-targeting therapies (BBI-608, BBI-503), chemotherapies (doxorubicin and gemcitabine), and a targeted therapy (sunitinib) with respect to A549 cells in the alginate-based 3D cell culture and the traditional 2D culture. As shown in FIG. 4A, the IC₅₀ values of the stemness inhibitors BBI-503 and BBI-608 are lower in the alginate-based 3D cell culture than those in the conventional 2D cultures, whereas the IC₅₀ values of sunitinib, doxorubicin, and gemcitabine are greater in the alginate-based 3D cell cultures than those in the conventional 2D cultures. To compare the effect of culturing cells in the alginate-based 3D and the conventional 2D cell cultures on IC₅₀, the fold change in resistance can be calculated by obtaining the ratio of IC₅₀ value in the alginate-based 3D cell culture to the IC₅₀ value in the conventional 2D culture. As shown in FIG. 4B, the chemotherapy and targeted therapy drugs have a fold change resistance greater than 1, thus confirming the drug resistance of the cells cultured in alginate-based 3D cell cultures of the present disclosure. Continuing to refer to FIG. 4B, cells in the alginate-based 3D cell culture treated with BBI-608 and BBI-503 have a change resistance less than single-fold, thus confirming that tumor cells, for example, the A549 cell line, are sensitive to stemness-targeting drugs in alginate-based 3D cell cultures of the present disclosure.

As shown in FIG. 1 to FIG. 4, single cells cultured in the alginate-based 3D cell cultures are less likely to form multicellular spheres when treated with the stemness-targeting drugs, as compared to cells treated with the non-stemness-targeting agents.

To evaluate the efficacy of the drugs in diffusion-limited environments, A549 cells were embedded and cultured for 11 days before any drug treatment (BBI-608, BBI-503, gemcitabine, or doxorubicin). Spheroids of A549 cells were pre-formed from single cells embedded in an alginate-based 3D cell culture for 11 days. The pre-formed spheroids were treated with drugs (0.3 μM BBI-608, 2.5 μM BBI-503, 30 μM sunitinib, 4 μM doxorubicin, 0.5 μM gemcitabine) and incubated with the dead stain CellTox Dye. Micrographs were taken 7 days after the drug treatments.

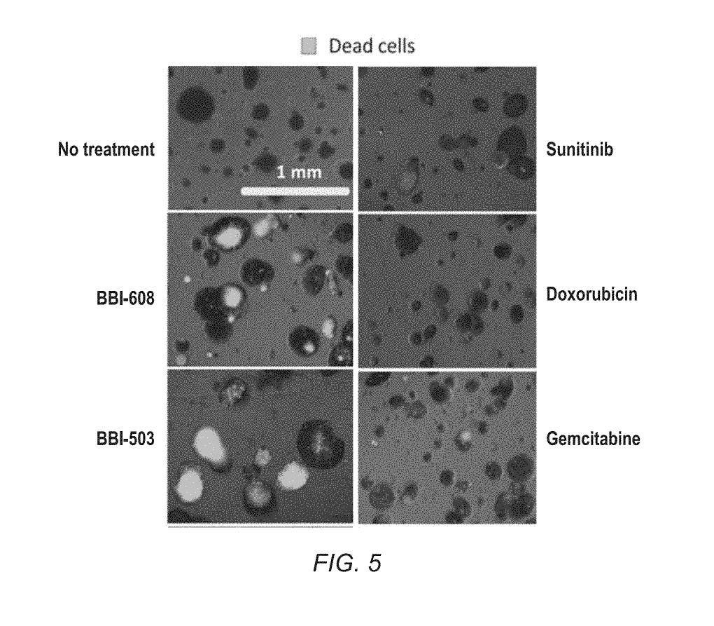

As shown in FIG. 5, administration of BBI-608 and BBI-503 at concentrations near their IC₅₀ values resulted in cell death in about 90% of the spheroids, while administration of doxorubicin and gemcitabine at concentrations near their IC₉₀ values resulted in cell death in no more than 40% of the spheroids. Without limiting the scope of the present disclosure or appended by any particular theory or hypothesis, these results indicated that: (i) cells in an alginate-based 3D cell culture of the present disclosure contain mostly those with high stemness, and (ii) BBI-608 and BBI-503 can efficiently kill these cells in the diffusion-limited spheroids, which likely contain cells with heterogeneous stemness properties.

### EXAMPLES

2-Acetylnaphtho[2,3-b]furan-4,9-dione or the compound of formula I may be synthesized, e.g., according to Examples 8-11 in U.S. Pat. No. 9,084,766. The compound of formula II may be synthesized, e.g., according to U.S. Pat. No. 8,299,106.

### Materials

Unless stated otherwise, cell culture media and reagents were purchased from ThermoFisher Scientific (Waltham, Mass.). A549 cells were obtained from American Type Culture Collection (Rockville, Md.). Adherent cells were maintained in Dulbecco's Modified Eagle Medium with 10% Fetal Bovine Serum (Gemini Bio-Products) and 1% Anti-Anti prior to suspension in alginate. Alginate (ProtanalLF 10/60FT) was obtained from FMC BioPolymer (Philadelphia, Pa.), silicone isolation sheet material (Product No. 644182) from Grace Bio-Labs, Inc. (Bend, Oreg.), VWR Blotting Paper 703 from VWR (Radnor, Pa.), and glass plates from BioRad (Hercules, Calif.). Sunitinib, Doxorubicin, and Gemcitabine were purchased from Selleckchem (Houston, Tex.).

### Example 1. Embedding and Culture of Cells in Alginate-Based 3D Cell Culture

Cells were suspended in a solution of alginate and basal media (0.6% v/v alginate in basal media). Unless stated otherwise, the cell suspension was prepared at a density of 5×10⁴ A549 cells/mL; these cells were either trypsinized from adherent cells, or dissociated using Accutase from cellular spheroids. For most of the experiments, the alginate solution was dispensed into a mold composed of a glass plate and silicone sheet, and then the mold was covered with sheets of blotting paper pre-soaked in crosslinking solution (100 mM CaCl₂, 10 mM HEPES, pH 7.4). The gels were incubated in the mold-paper construct at 37° C. for 15 minutes resulting in a complete gelling. The alginate gelled and embedded the cells as the cross-linking solution diffused from the blotting papers into the solution of alginate in the mold. For each passage, the cells were cultured between 7 and 14 days in Prime XV media (Irvine Scientific, Irvine, Calif.) supplemented with B27 and 1% Anti-Anti. The embedded cells grew from single cells to cellular spheroids during the culture. To release the spheroids from the alginate-based 3D cell culture, the cell-embedded gels were incubated in a dissolution buffer (10 mM EDTA, 5 mM citrate, 150 mM NaCl, pH 7.0) for 10 minutes at room temperature on a benchtop shaker. The cellular spheroids were washed with PBS and then pelleted by centrifugation. The pellets were either treated with Accutase to dissociate the spheroids into single cells for succeeding passages, or frozen for future analyses.

### Example 2. Evaluation of Stemness and Glucose Metabolism Properties

Total RNA was extracted from the frozen cell pellets using a Maxwell® RSC simplyRNA Kit and a Maxwell® RSC Instrument (Promega, Madison, Wis.), and the concentration of the RNA from each sample was determined using the Nanodrop 1000 Spectrophotometer (Thermo Scientific, Waltham, Mass.). Reverse transcription of RNA and synthesis of cDNA using 1.50 μg of RNA per sample was performed with iScript™ Reverse Transcription Supermix for RT-qPCR (Bio-Rad) according to the manufacturer's instructions. Cancer Stem Cells (SAB Target List) H96 and Glucose metabolism (SAB Target List) H96 qPCR arrays (BioRad) were used to evaluate the expression of CSC-related genes and glucose-metabolism genes, respectively. Following the manufacturer's protocol, 8.33 ng cDNA per well was used for both types of array. Expression of CSC-related, hypoxia-related, and glycolysis related genes were validated using qPCR. qPCR-based gene validation was carried out using 1 μg cDNA per sample with iTaq™ Universal SYBR® Green Supermix (BioRad) according to the manufacturer's protocol.

### Example 3. Sphere Formation Assay

Into each well of a flat bottom-96 well plate, 30 μL suspension of the cells in alginate solution was dispensed. 9 μL of crosslinking solution was then added to each well to form alginate gels and thus embed A549 cells into the gel matrix. The plates were then incubated for 15 minutes at 37° C. to ensure a complete gelling of alginate, and then cultured in Prime XV media overnight. After the overnight culture, the cells were treated with various concentrations of BBI-608, BBI-503, sunitinib, doxorubicin, or gemcitabine for 24 h. The plates were then washed and cultured in fresh media for an additional 11 days before imaging the cultures with Celigo Imaging Cytometer (Nexcelcom Bioscience, Lawrence, Mass.). The number of spheres were counted, and used to calculate the IC₅₀ values using Prism 5 (GraphPad).

### Example 4. Clonogenic Assay

A549 cells were plated in 6-well plates (1×10³ A549 cells/well). After an overnight incubation in DMEM media, the cells were treated with various concentrations of BBI-608 for 24h, or BBI-503, sunitinib, doxorubicin, and gemcitabine for 72 hours. The plates were washed, and cultured in fresh media for an additional 6 days before staining the colonies with Giemsa Stain (Sigma, St. Louis, Mo.). The colonies were imaged using a ChemiDoc™ MP Imaging System (BioRad), and the number of colonies were counted using ImageJ (National Institutes of Health, Bethsada, Md.), and the IC₅₀ values calculated using Prism 5.

### Example 5. Efficacy of Chemotherapy Drugs of Pre-Formed Cellular Spheroids Cultured in Alginate-Based 3D Cell Culture

In a 6-well plate, A549 cells were embedded in an alginate-based 3D cell culture and cultured for 11 days before addition of BBI-608, BBI-503, sunitinib, doxorubicin, and gemcitabine. Cells were cultured for an additional 7 days before staining them with CellTox™ Green Dye (Promega) to stain the dead cells. Images of the stained samples were taken using an EVOS fluorescence microscope (Life Technologies, Carlsbad, Calif.).

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described; accordingly, all suitable modifications and equivalents may be resorted to, falling within the scope of the present disclosure.

Moreover, those of ordinary skill in the art will appreciate that the conception upon which this disclosure is based may readily be used as a basis for designing other pharmaceutical compositions and pharmaceutical tablets for carrying out the several purposes of the present disclosure. Accordingly, the claims are not intended to be limited by the foregoing description.

1. A method of treating cells to provide cells enriched with at least one stem-like characteristic comprising embedding cells in a cell culture, wherein the cell culture comprises at least one alginate and at least one crosslinker, and wherein the cells are enriched with at least one stem-like characteristic.
